(19) United States
(12) Patent Application Publication
(10) Pub. No.: US 2008/0145942 A1
(43) Pub. Date: Jun. 19, 2008

(54) METAL DETECTION REAGENTS INCLUDING AN AMMONIUM SALT AND METHODS OF USING THE SAME

(75) Inventors: Hyun-Kee Hong, Daejeon (KR); Jae-Seok Lee, Seoul (KR); Yang-Koo Lee, Gyeonggi-do (KR); Hun-Jung Yi, Gyeonggi-do (KR)

Correspondence Address: MYERS BIGEL SIBLEY & SAJOVEC, PO BOX 37428, RALEIGH, NC, 27627, US

(73) Assignee: Samsung Electronics Co., Ltd.

(21) Appl. No.: 11/955,086

(22) Filed: Dec. 12, 2007

(30) Foreign Application Priority Data
Dec. 13, 2006 (KR) 10-2006-0126927

(51) Int. Cl. G01N 33/20

(52) U.S. Cl. 436/73

(57) ABSTRACT

Provided herein are metal detection reagents including at least one ammonium salt of Formula 1:

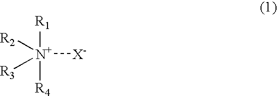

wherein R₁, R₂, R₃ and R₄ are independently selected from the group consisting of hydrogen, C₁₋₃₀ alkyl and C₃₋₁₄ aryl, and X⁻ is independently selected from the group consisting of bromide, chloride, iodide, fluoride, nitrate, phosphate and sulfate and methods of using the metal detection reagents to monitor one or more metal ion levels in a solution.

### CROSS-REFERENCE TO RELATED APPLICATIONS

This application claims priority to Korean Patent Application No. 2006-126927, filed Dec. 13, 2006, the disclosure of which is hereby incorporated by reference in its entirety.

### FIELD OF THE INVENTION

Embodiments of the present invention relate to metal detection reagents and methods of using the same. In particular, exemplary embodiments of the present invention relate to metal detection reagents and methods of monitoring a cleaning solution using the metal detection reagents.

### BACKGROUND OF THE INVENTION

A semiconductor device may be contaminated by metal, for example, aluminum or iron, during a semiconductor manufacturing process. Metal contamination may affect the manufacturing yield and the operational characteristics of a semiconductor device. Since semiconductor devices have become highly integrated, there is a need to control metal contamination during the manufacturing process. Therefore, research has been conducted to study the impact of metal contamination during the semiconductor manufacturing process and methods of controlling metal contamination.

Studies reveal that metal contamination may increase the possibility of leakage current of a reverse bias junction, and thus may adversely affect the characteristics of a semiconductor device such as dielectric breakdown strength, capacitance and leakage current. Moreover, metal contaminants may react with silicon or silicon oxide to form metal silicide or metal silicate, which may cause deterioration of a semiconductor device. Therefore, a cleaning process may be performed between different steps of semiconductor manufacturing processes to decrease metal contamination.

Metal contamination may be formed in processes such as an etching process, chemical mechanical polishing process and ion implantation process. These processes may be carried out on a wafer, which is cleaned by a cleaning solution in a cleaning bath. After several wafers are cleaned, the cleaning solution may be significantly contaminated by metal ions, and the metal contaminants may accumulate inside the cleaning bath. Therefore, a wafer may be re-contaminated by the cleaning solution. For example, a cleaning solution (SC-1) including a mixture of NH₄OH, H₂O₂ and H₂O in a volume ratio of about 1:4:20 may be used to remove impurities from a wafer. During the cleaning process, the metal impurities in the SC-1 may migrate to the wafer. In addition, in some situations, a cleaning solution including diluted HF (DHF) may react with metal contaminants to form an unstable metal fluoride complex. The fluoride complex may initiate an oxidation/reduction reaction on the surface of a wafer, which may cause a defect on the wafer surface.

Korean Patent No. 0585139 (hereinafter the '139 Korean Patent) discusses a method of monitoring contamination of a cleaning solution by using an azo compound as a chelating agent to detect a metal ion in a cleaning solution. The metal detection reagent in the '139 Korean Patent may detect a metal ion such as aluminum ion when the concentration of the metal ion is above 50 ppb. Since semiconductor devices have become highly integrated, it is desirable to maintain a low concentration of the metal ion in the cleaning solution. Therefore, there is a need to develop a metal detection reagent to detect a low concentration of metal ion.

### SUMMARY OF THE INVENTION

According to some embodiments of the present invention, methods of monitoring the level of one or more metal ions in a solution include:

preparing a metal detection reagent including at least one ammonium salt of Formula 1, a chelating agent and a solvent, wherein Formula 1 has the following structure:

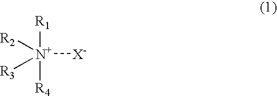

wherein R₁, R₂, R₃ and R₄ are independently selected from the group consisting of hydrogen, C₁₋₃₀ alkyl , C₃₋₁₀ cycloalkyl, C₃₋₁₄ aryl, and C₃₋₁₄ heteroaryl, and X⁻ is selected from the group consisting of bromide, chloride, iodide, fluoride, nitrate, phosphate and sulfate;

adding the metal detection reagent to the solution to form a mixture; and

monitoring the level of one or more metal ions in the solution by measuring absorbance of the mixture.

In some embodiments of the present invention, the metal ion is detected by measuring a maximum absorbance of the mixture at a wavelength in a range of about 560 nm to about 650 nm. In some embodiments, the mixture of the metal detection reagent and the solution has a pH from about 4.8 to about 7.5.

Further, in some embodiments, the metal ions are selected from the group consisting of aluminum and transition metals. In some embodiments, the metal ions are selected from the group consisting of nickel, iron, copper, cobalt, manganese, zinc, lead and platinum. Yet, in another embodiment, the metal ion is aluminum.

In some embodiments, monitoring the level of one or more metal ions is carried out using a variable-wavelength UV-Vis detector.

Another aspect of the present invention relates to metal detection reagents including at least one ammonium salt of Formula 1:

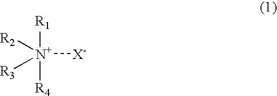

wherein R₁, R₂, R₃ and R₄ are independently selected from the group consisting of hydrogen, C₁₋₃₀ alkyl, C₃₋₁₀ alkyl, C₃₋₁₄ aryl, C₃₋₁₄ heteroaryl, and X⁻ is independently selected from the group consisting of bromide, chloride, iodide, fluoride, nitrate, phosphate and sulfate; a chelating agent and a solvent. In some embodiments, the solvent is water.

In some embodiments, the weight ratio of the ammonium salt to the chelating agent is about 1:1 to about 30:1. Further, in some embodiments, the ammonium salt includes at least one compound selected from the group consisting of cetyltrimethyl ammonium bromide, octadecyltrimethyl ammonium bromide, pentadecyltrimethyl ammonium chloride, dodecyltrimethyl ammonium chloride, decyltrimethyl ammonium bromide, octyltrimethyl ammonium bromide, hexyltrimethyl ammonium bromide, butyltrimethyl ammonium chloride, benzyltrimethyl ammonium chloride, diethyldimethyl ammonium chloride, dioctyldimethyl ammonium bromide, tetrabutyl ammonium chloride, tetrapropyl ammonium chloride, tetraethyl ammonium chloride, tetramethyl ammonium chloride, and cetyltrimethyl ammonium phosphate and a mixture thereof. In some embodiments of the present invention, the ammonium salt is cetyltrimethyl ammonium bromide.

In some embodiments, the chelating agent is selected from the group consisting of eriochrome cyanine R (ECR), chrome azurol-S, 8-hydroxyquinoline derivatives, 1,2-dihydroxy-3,5-benzene disulfonic acid disodium salt (tiron), hydroxy-2-(2-hydroxyphenylazo)benzene, 5-chloro-2-hydroxy-3-(2,4-dihydroxyphenylazo)benzenesulfonic acid (lumogallion), pyrocatechol violet (PV) and a mixture thereof. In some embodiments of the present invention, the chelating agent is eriochrome cyanine R. In some embodiments, the weight percentage of the chelating agent to the metal detection reagent is in a range of about 0.0001 to about 0.01 percent. In some embodiments, the weight percentage of the chelating agent to the metal detection reagent is in a range of about 0.001 to about 0.01 percent. In some embodiments, the weight percentage of the chelating agent to the metal detection agent is in a range of about 0.002 to about 0.01 percent.

Further, in some embodiments, the weight percentage of the ammonium salt to the metal detection agent is in a range of about 0.00001 to about 1 percent and the weight ratio of the chelating agent to the metal detection agent is in a range of about 0.000001 to about 1 percent. In some embodiments, the ammonium salt is in the range of about 0.00001 to about 1 percent by weight, the chelating agent is in a range of about 0.000001 to about 1 percent by weight, and the pH-controlling agent is in a range of about 1 to about 30 percent by weight. In some embodiments, the weight percentage of the ammonium salt to the metal detection agent is in a range of about 0.001 to about 0.1 percent. In some embodiments, the weight percentage of the ammonium salt to the metal detection agent is in a range of about 0.002 to about 0.01 percent. In some embodiments, the amount of the ammonium salt is substantially equal to or greater than the chelating agent.

In certain embodiments, the metal detection reagent further includes a pH-controlling agent. In some embodiments, the pH-controlling agent is selected from the group consisting of acetic acid, citric acid, sulfuric acid, nitric acid, hydrochloric acid, ammonium hydroxide, sodium hydroxide and potassium hydroxide or a mixture thereof. In some embodiments, the metal detection reagent has a pH in a range of about 1 to about 3, when the pH-controlling agent is acidic. In another embodiment, the metal detection reagent has a pH in a range of about 8 to about 10 when the pH-controlling agent is basic.

### BRIEF DESCRIPTION OF THE DRAWINGS

The foregoing and other objects, features and advantages of the invention will become more apparent from the following more particular description of exemplary embodiments of the invention and the accompanying drawings. The drawings are not necessarily to scale, emphasis instead being placed upon illustrating the principles of the invention.

FIG. 1 is a flow chart illustrating a method of monitoring a cleaning solution according to some embodiments of the present invention.

FIG. 2 is a schematic diagram illustrating an apparatus employed in a method of monitoring a cleaning solution according to some embodiments of the present invention.

FIG. 3 graphically illustrates absorbance of aluminum complexes using the metal detection reagents prepared in Example 1 and Comparative Example 1.

FIGS. 4 and 5 graphically illustrate the variation of maximum absorbance of aluminum complexes in the metal detection reagents of Example 1 and Comparative Example 1 over time.

FIGS. 6 and 7 graphically illustrate the variation of maximum absorbance of aluminum complexes in the metal detection reagents of Example 1 and Comparative Example 1 over the concentration of aluminum ion.

The present invention will now be described more fully hereinafter with reference to the accompanying drawings, in which embodiments of the invention are shown. This invention may, however, be embodied in many different forms and should not be construed as being limited to the embodiments set forth herein. Rather, these embodiments are provided so that this disclosure will be thorough and complete, and will fully convey the scope of the invention to those skilled in the art.

### DESCRIPTION OF THE EMBODIMENTS

The terminology used in the description of the invention herein is for the purpose of describing particular embodiments only and is not intended to be limiting of the invention. As used in the description of the embodiments of the invention and the appended claims, the singular forms “a,” “an” and “the” are intended to include the plural forms as well, unless the context clearly indicates otherwise. Also, as used herein “and/or” refers to and encompasses any and all possible combinations of one or more of the associated listed items.

Unless otherwise defined, all terms, including technical and scientific terms used in this description, have the same meaning as commonly understood by one of ordinary skill in the art to which this invention belongs. All publications, patent applications, patents, and other references mentioned herein are incorporated by reference in their entirety.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Moreover, it will be understood that steps comprising the methods provided herein can be performed independently or at least two steps can be combined. Additionally, steps comprising the methods provided herein, when performed independently or combined, can be performed at the same temperature and/or atmospheric pressure or at different temperatures and/or atmospheric pressures without departing from the teachings of the present invention.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

As will be appreciated by one of ordinary skill in the art, the present invention may be embodied as compositions and devices including the compositions as well as methods of making and using such compositions and devices.

### DEFINITIONS

“Alkyl” as used herein, means a straight-chain, (i.e., unbranched), branched, or cyclic hydrocarbon chain that is completely saturated. In certain embodiments, alkyl groups contain 1 to 30 carbon atoms. In some embodiments, alkyl groups contain 1-20 carbon atoms. In some embodiments, alkyl groups contain 1 to 10 carbon atoms, and yet in other embodiments, 1-6 carbon atoms. In other embodiments, alkyl groups contain 1 to 3 carbon atoms. In still other embodiments, alkyl groups contain 2-5 carbon atoms, and in yet other embodiments, alkyl groups contain 1-2, or 2-3 carbon atoms. In certain embodiments, the term “alkyl” refers to a cycloalkyl group, also known as carbocycle. Exemplary C₁₋₃ alkyl groups include methyl, ethyl, propyl, isopropyl, and cyclopropyl.

“Cycloalkyl,” as used herein alone or as part of another group, refers to groups having 3 to 10 carbon atoms. In some embodiments, the cycloalkyl employed in the invention have 3 to 8 carbon atoms. Suitable cycloalkyls include, but are not limited to, cyclopropyl, cyclobutyl, cyclopentyl, cyclohexyl, cycloheptyl and the like. The “cycloalkyl” can be unsustituted or substituted with chemically feasible substituents.

“Aryl” as used herein alone or as part of another group, refers to a monocyclic carbocyclic ring system or a bicyclic carbocyclic fused ring system having one or more aromatic rings. In some embodiments, the aryl may have 3 to 14 carbon atoms. Representative examples of aryl include, azulenyl, indanyl, indenyl, naphthyl, phenyl, tetrahydronaphthyl, and the like. The term “aryl” can be unsubstituted or substituted with chemically feasible substituents.

“Heteroaryl” as used herein alone or as part of another group, refers to a cyclic, aromatic hydrocarbon in which one or more carbon atoms have been replaced with heteroatoms such as O, N, and S. If the heteroaryl group contains more than one heteroatom, the heteroatoms may be the same or different. In some embodiments, the heteroaryl employed in the invention have 3 to 14 carbon atoms. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. In some embodiments, heteroaryl groups are five and six membered rings and contain from one to three heteroatoms independently selected from O, N, and S. The heteroaryl group, including each heteroatom, can be unsubstituted or substituted with chemically feasible substituents. For example, the heteroatom N or S may be substituted with one or two oxo groups, which may be shown as ═O.

### I. Metal Detection Reagent

A metal detection reagent according to some embodiments of the present invention includes at least one ammonium salt. In some embodiments, the metal detection agent may further include a chelating agent and a solvent such as water.

In some embodiments, the chelating agent may react with a metal ion to form a metal complex, which may absorb light at a visible wavelength, which is about 400 to about 700 nanometers (nm). In some embodiments, the chelating agent may be selected from the group consisting of eriochrome cyanine R (ECR), chrome azurol-S, 8-hydroxyquinoline derivatives, 1,2-dihydroxy-3,5-benzene disulfonic acid disodium salt (tiron), hydroxy-2-(2-hydroxyphenylazo)benzene, 5-chloro-2-hydroxy-3-(2,4-dihydroxyphenylazo)benzenesulfonic acid (lumogallion), pyrocatechol violet (PV) and a mixture thereof. In some embodiments, the chelating agent may include eriochrome cyanine R.

In some embodiments of the present invention, the weight percentage of the chelating agent to the metal detection agent may be in a range of about 0.000001 to about 1 percent. In some embodiments of the present invention, the weight percentage is in a range of about 0.0001 to about 0.01 percent. In another embodiment, the weight percentage is in a range of about 0.001 to about 0.01 percent. In some embodiments, the weight percentage of the chelating agent to the metal detection agent is in a range from about 0.002 to about 0.01 percent. In further embodiments, the weight percentage of the ammonium salt to the metal detection agent is in a range of about 0.00001 to about 1 percent and the weight percentage of the chelating agent to the metal detection agent is in a range of about 0.000001 to about 1 percent.

In some embodiments of the present invention, the ammonium salt may associate with the chelating agent to form a metal complex. In further embodiments of the present invention, the ammonium salt may react with a metal ion and the chelating agent to form a metal complex. Because of the presence of the ammonium salt, the wavelength of the metal complex may be in a lower ground absorbance.

In some embodiments of the present invention, the ammonium salt is represented by Formula 1:

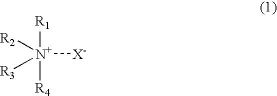

wherein R₁, R₂, R₃ and R₄ are independently selected from the group consisting of hydrogen, C₁₋₃₀ alkyl, C₃₋₁₀alkyl, C₃₋₁₄ aryl, C₃₋₁₄heteroaryl, and X⁻ is independently selected from the group consisting of bromide, chloride, iodide, fluoride, nitrate, phosphate and sulfate.

In some embodiments of the present invention, the ammonium salt may be selected from the group consisting of cetyltrimethyl ammonium bromide, octadecyltrimethyl ammonium bromide, pentadecyltrimethyl ammonium chloride, dodecyltrimethyl ammonium chloride, decyltrimethyl ammonium bromide, octyltrimethyl ammonium bromide, hexyltrimethyl ammonium bromide, butyltrimethyl ammonium chloride, benzyltrimethyl ammonium chloride, diethyldimethyl ammonium chloride, dioctyldimethyl ammonium bromide, tetrabutyl ammonium chloride, tetrapropyl ammonium chloride, tetraethyl ammonium chloride, tetramethyl ammonium chloride, cetyltrimethyl ammonium phosphate and a mixture thereof. In another embodiment of the present invention, the ammonium salt may be cetyltrimethyl ammonium bromide.

In some embodiments of the present invention, the weight percentage of the ammonium salt to the metal detection agent may be in a range from about 0.00001 to about 1 percent. In another embodiment of the present invention, the weight percentage may be in a range from about 0.001 to about 0.1 percent. In some embodiment of the present invention, the weight percentage is in a range from about 0.002 to about 0.01 percent.

In some embodiments of the present invention, the amount of the ammonium salt may be substantially equal to or greater than the chelating agent. In another embodiment, the weight ratio of the ammonium salt and the chelating agent may be about 1:1 to about 30:1.

In some embodiments of the present invention, the metal detection reagent may further include a pH-controlling agent. The pH-controlling agent may include an acid and/or a base. In some embodiments of the present invention, the pH-controlling agent may be selected from the group consisting of acetic acid, citric acid, sulfuric acid, nitric acid, hydrochloric acid, ammonium hydroxide, sodium hydroxide, potassium hydroxide and a mixture thereof. When the cleaning solution is basic, the pH-controlling agent may include an acid. When the cleaning solution is acidic, the pH-controlling agent may include a base. When the pH-controlling agent is acidic, the metal detection reagent has a pH in a range from about 1 to about 3. When the pH-controlling agent is basic, the metal detection reagent has a pH in a range from about 8 to about 10

In some embodiments of the present invention, the metal detection reagent may mix with a cleaning solution containing a metal ion to form a metal complex which may absorb visible light. In some embodiments, the wavelength of the metal complex may be in a range of about 560 nm to about 650 nm. In another embodiment of the present invention, the metal ion in the cleaning solution may be an aluminum ion.

### II. Methods of Monitoring a Cleaning Solution

Methods of monitoring a cleaning solution will now be described in detail with reference to the accompanying drawings.

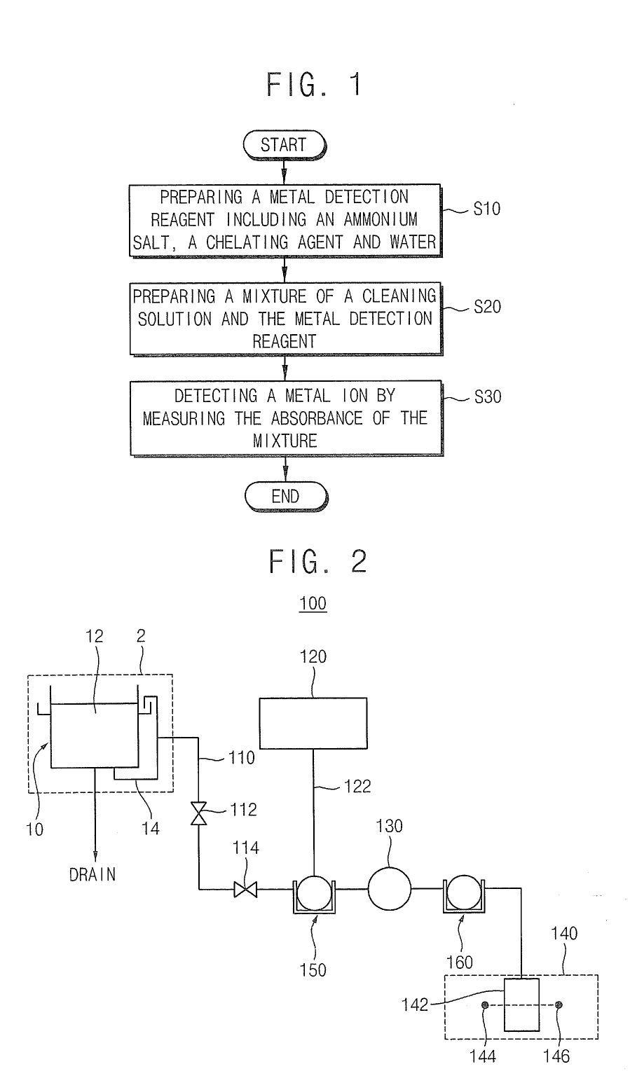

FIG. 1 is a flowchart illustrating the method of monitoring a cleaning solution according to some embodiments of the present invention.

Referring to FIG. 1, step S10 is directed to preparing a metal detection reagent including an ammonium salt of Formula 1, a chelating agent and water.

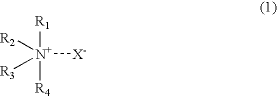

In Formula 1, R₁, R₂, R₃ and R₄ are independently selected from the group consisting of hydrogen, C₁₋₃₀ alkyl, C₃₋₁₀cycloalkyl, C₃₋₁₄ aryl and C₃₋₁₄ heteroaryl, and X⁻ is selected from the group consisting of bromide, chloride, iodide, fluoride, nitrate, phosphate and sulfate. The metal detection reagent may further include a pH-controlling agent as described above.

Step S20 is directed to preparing a mixture by adding the metal detection reagent to a solution such as a cleaning solution. A metal ion in the cleaning solution may react with the chelating agent and the ammonium salt to form a metal complex.

The cleaning solution may be any cleaning solution used in the manufacturing process of a semiconductor device. For example, the cleaning solution may be NH₄OH/H₂O₂/H₂O (hereinafter “SC-1”), choline/H₂O₂/H₂O, H₂SO₄/H₂O₂, HCl/H₂O₂/H₂O, HNO₃/HF/H₂O, H₂SO₄/H₂O₂, NH₄OH/H₂O₂/H₂O, HF/H₂O₂, HF/NH₄F/H₂O, diluted HF (hereinafter “DHF”), HF/NH₄F, HF/HNO₃/CH₃COOH, H₃PO₄, HNO₃/H₃PO₄/CH₃COOH, or water. Exemplary metal ions to be detected may be selected from the group consisting of aluminum, transition metals such as nickel, iron, copper, cobalt, manganese, zinc, lead and platinum.

In some embodiments of the present invention, the mixture of the cleaning solution and the metal detection reagent may have a pH in the range of about 4.8 to about 7.5. An acidic or basic pH-controlling agent may be added to the metal detection reagent to adjust the pH to the above range. For example, if the cleaning solution is basic, an acidic pH-controlling agent such as an organic acid or an inorganic acid may be added to adjust the pH of the mixture of cleaning solution and the metal detection agent. If the cleaning solution is acidic, a basic pH-controlling agent such as ammonium hydroxide, sodium hydroxide, or potassium hydroxide, may be added to adjust the pH of the mixture of the cleaning solution and the metal detection agent.

Step S30 is directed to measuring absorbance of the mixture of the cleaning solution and the metal detection agent to monitor one or more metal ion levels in the cleaning solution. The absorbance may be measured by an ultraviolet-visible (UV-Vis) spectrometer. A metal complex may be formed by a metal ion, the chelating agent and/or the ammonium salt in the cleaning solution, and it may have a maximum absorbance at a wavelength of visible light. For example, if aluminum exists in the cleaning solution, the aluminum complex may have a maximum absorbance at a wavelength in the range of about 560 nm to about 650 nm. The absorbance noise originated from the metal detection reagent and/or the cleaning solution may be negligible in the wavelength of visible light. Therefore, the metal detection agent may be able to detect metal ions at a low concentration.

FIG. 2 is a schematic diagram illustrating an apparatus employed in the method of monitoring a cleaning solution according to some embodiments of the present invention.

Referring to FIG. 2, an apparatus 100 for monitoring metal contamination includes a sampling line 110, a reagent reservoir 120, a mixer 130, and an absorption measuring device 140. The sampling line 110 delivers cleaning solution 12 from a cleaning bath 10 in a wet station 2. The reagent reservoir 120 contains a metal detection reagent. The mixer 130 is used to mix the cleaning solution 12 and the metal detection reagent. The absorption measuring device 140 measures the absorbance of the mixture from the mixer 130, and the measured absorbance may be displayed by a monitor. The absorption measuring device 140 may include a UV-Vis spectrometer or a variable-wavelength UV-Vis detector. For example, the absorption measuring device 140 may include a UV cell 142 to introduce the mixture, a light source 144 and a detector 146 to measure the absorbance of the mixture. Since the variable-wavelength UV-Vis detector may monitor two or more wavelengths at the same time, it may detect and analyze two or more metal complexes simultaneously. Therefore, using variable-wavelength UV-Vis detector may improve sensitivity and/or selectivity for detecting different types of metal complexes.

According to some embodiments of the present invention, the wet station 2 may include a circulation line 14 to circulate the cleaning solution in the cleaning bath 10. The sampling line 110 may be connected to the circulation line 14. A reagent supply line 122 provides the metal detection reagent in the reagent reservoir 120 to the mixer 130. In some embodiments, a pump 150 may be positioned between the reagent supply line 122 and the mixer 130 to control the flow rate of the metal detection reagent. In certain embodiments, a second pump 160 may be positioned between the mixer 130 and the absorption measuring device 140 to further control the flow rate of the mixture of the metal detection reagent and the cleaning solution. In some embodiments, the sampling line 110 may have needle valves 112 and 114 to adjust the flow rate of the cleaning solution 12.

In some embodiments, the mixture in the mixer 130 is prepared by a predetermined weight or volume ratio of the cleaning solution 12 and the metal detection reagent. The pH of the mixture in the mixer 130 may be in a range from about 4.8 to about 7.5. The mixture in the mixer 130 may be provided to the absorption measuring device 140 to measure the absorbance of the mixture. In some embodiments, the absorbance of the mixture may be measured at a wavelength of a visible light. In some embodiments, the metal ion is detected by measuring a maximum absorbance of the mixture at a wavelength in a range of about 560 nm to about 650 nm. In another embodiment, cleaning the wafer in the cleaning bath 10, sampling the cleaning solution 12, mixing the cleaning solution and the metal detection reagent, and measuring the absorbance of the mixture may be carried out at the same time.

### EXAMPLES

### Preparation of Metal Detection Reagents

### Example 1

A metal detection reagent was prepared by mixing about 0.25 g eriochrome cyanine R (ECR), about 0.75 g cetyltrimethyl ammonium bromide (CTAB), and about 10 L of water. The pH of the metal detection reagent was at about 6.0.

### Comparative Example 1

A metal detection reagent was prepared by mixing about 0.25 g eriochrome cyanine R (ECR) and about 10 L water. The pH of the metal detection reagent was about 6.0. The amount of each component used in preparing the metal detection reagent is shown in Table 1.

TABLE 1

| | ECR [g] | Ammonium Salt [g] | Water [L] |
|---|---|---|---|
| Example 1 | 0.25 | 0.75 | 10 |
| Comparative Example 1 | 0.25 | — | 10 |

Absorbance of an Aluminum Complex

An aluminum ion was added to the metal detection reagents prepared in Example 1 and Comparative Example to form an aluminum complex, and then the absorbance of the complex was measured. The concentration of the aluminum ion was about 10 ppb.

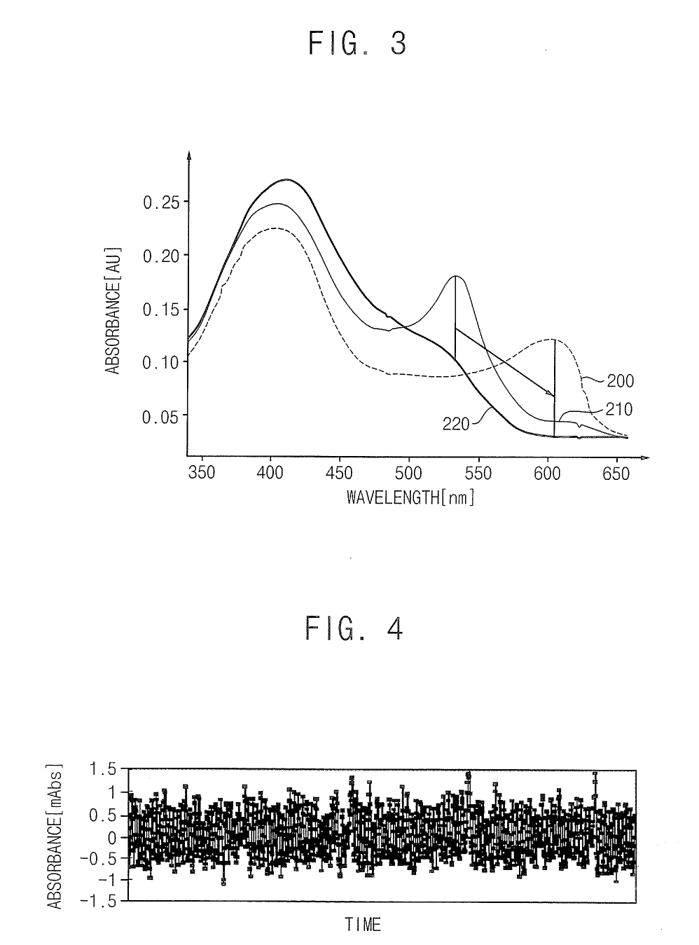

FIG. 3 is a graph illustrating absorbance of aluminum complexes generated in Example 1 and Comparative Example 1. In FIG. 3, the first graph line 200 demonstrates the absorbance of the aluminum complex in Example 1, a second graph line 210 demonstrates the absorbance of the aluminum complex in Comparative Example 1, and a third graph line 220 shows the absorbance of the metal detection reagent of Example 1.

Referring to FIG. 3, the aluminum complex in Example 1 had a maximum absorbance at a wavelength of about 610 nm. The aluminum complex in Comparative Example 1 had a maximum absorbance at a wavelength of about 540 nm. By comparison, the ground absorbance of the metal detection reagent was relatively high at a wavelength of about 540 nm and low at a wavelength of about 610 nm.

The shift of the wavelength may be caused by the association of the ammonium salt and the metal complex. Thus, compared to the conventional reagent, the metal detection reagent with the ammonium salt may enhance the sensitivity of detecting a metal ion since the ground absorbance of the metal detection agent is low at the absorbance wavelength of the metal ion.

Evaluation of Absorbance Noise

The maximum absorbance of aluminum complexes of the metal detection agents in Example 1 and Comparative Example 1 was measured. The concentration of the aluminum ion was about 10 ppb.

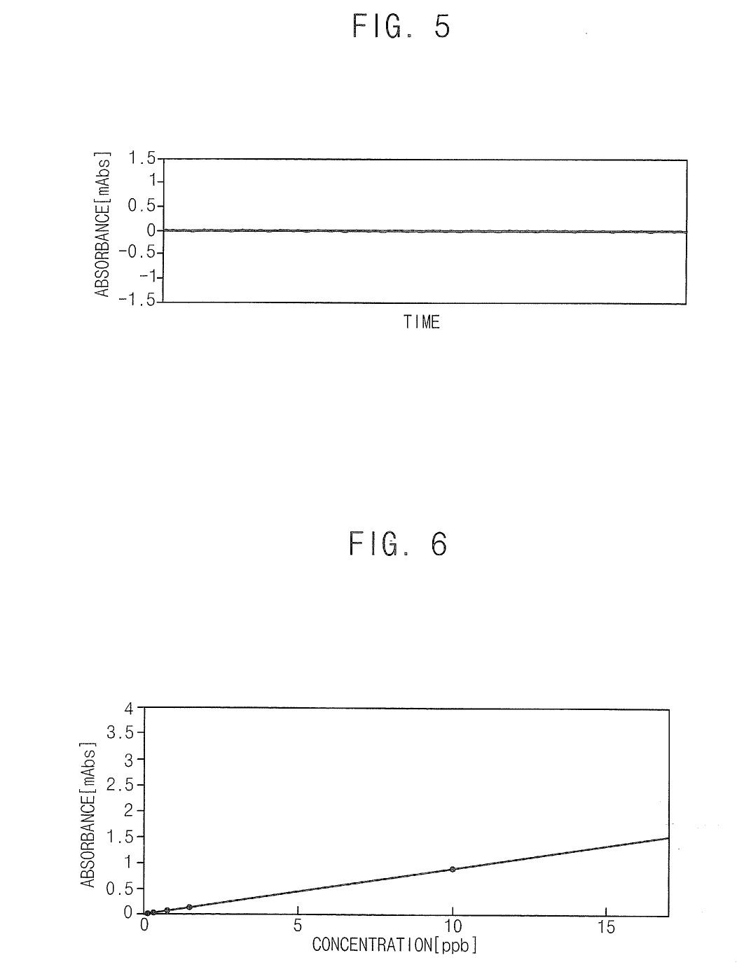

FIG. 4 illustrates the maximum absorbance of an aluminum complex varying over time in Comparative Example 1. FIG. 5 illustrates the maximum absorbance of an aluminum complex varying over time in Example 1. In FIG. 4, the maximum absorbance of the aluminum complex in Comparative Example 1 was measured at a wavelength of about 540 nm. In FIG. 5, the maximum absorbance of the aluminum complex in Comparative Example 1 was measured at a wavelength of about 610 nm.

Referring to FIG. 4, the maximum absorbance of the aluminum complex in Comparative Example 1 greatly varies over time, and there is serious absorbance noise. Referring to FIG. 5, the maximum absorbance of the aluminum complex in Example 1 varies little over time, and there is negligible absorbance noise. In FIG. 5, the absorbance noise at wavelength of about 610 nm was at least about fifty times less than that of 540 nm. Thus, comparing to the conventional reagent without the ammonium salt, the metal detection reagent including the ammonium salt may reduce absorbance noise.

Evaluation of Reactivity to Aluminum

The reactivity of the metal detection reagents in Example 1 and Comparative Example 1 to aluminum was evaluated.

Different concentrations of aluminum ion were added to the metal detection reagents to evaluate the reactivity of the metal detection agent to aluminum. The concentration was adjusted to about 0 ppb, about 0.2 ppb, about 0.5 ppb, about 1.5 ppb and about 10 ppb, and the maximum absorbance of an aluminum complex was measured at each concentration.

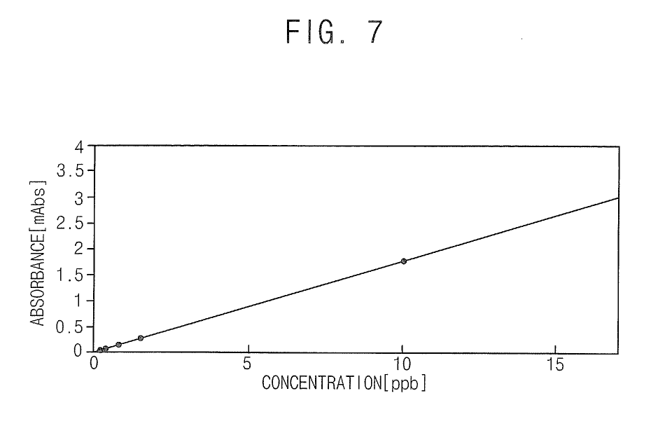

FIGS. 6 and 7 are graphs illustrating variation of a maximum absorbance over different concentrations of aluminum ion in Example 1 and Comparative Example 1.

Referring to FIGS. 6 and 7, when the concentration of aluminum is at 10 ppb, the metal detection reagent in Example 1 has a maximum absorbance about two times greater than that of the metal detection reagent in Comparative Example 1. Thus, the metal detection reagent in Example 1 has a higher sensitivity of aluminum than that of the metal detection reagent in Comparative Example 1. Therefore, in the exemplary embodiment, the metal detection reagent including the ammonium salt may detect a metal ion at a lower concentration.

According to some embodiments of the present invention, the absorbance of the metal complex formed by a metal detection reagent and metal ion may be in a wavelength range where the absorbance noise is negligible. Therefore, the metal detection reagent may detect a metal ion at a low concentration.

The foregoing is illustrative of the present invention and is not to be construed as limiting thereof. Although some embodiments of this invention have been described, one of ordinary skill in the art will readily appreciate that modifications to the embodiments are possible without departing from the teachings of the invention. Accordingly, all such modifications are intended to be included within the scope of this invention as defined in the claims, with equivalents of the claims to be included therein. In the claims, means-plus-function clauses are intended to cover structures described herein as performing the recited function and not only structural equivalents but also equivalent structures. Accordingly, it is understood that the foregoing is illustrative of the present invention and is not to be construed as limited to the specific embodiments disclosed, and that modifications to the disclosed embodiments, as well as other embodiments, are intended to be included within the scope of the claims.

What is claimed is:

1. A method of monitoring the level of one or more metal ions in a solution comprising:
preparing a metal detection reagent comprising at least one ammonium salt of Formula 1, a chelating agent and a solvent, wherein Formula 1 has the following structure 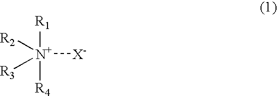
wherein R₁, R₂, R₃ and R₄ are independently selected from the group consisting of hydrogen, C₁₋₃₀ alkyl, C₃₋₁₀ cycloalkyl, C₃₋₁₄ aryl, and C₃₋₁₄ heteroaryl, and
X⁻ is selected from the group consisting of bromide, chloride, iodide, fluoride, nitrate, phosphate and sulfate;
adding the metal detection reagent to the solution to form a mixture; and
monitoring the level of one or more metal ions in the solution by measuring absorbance of the mixture.

2. The method of claim 1, wherein the metal ion is detected by measuring a maximum absorbance of the mixture at a wavelength in a range of about 560 nm to about 650 nm.

3. The method of claim 1, wherein the mixture has a pH from about 4.8 to about 7.5.

4. The method of claim 1, wherein the metal ions are selected from the group consisting of aluminum and transition metals.

5. The method of claim 1, wherein the metal ions are selected from the group consisting of nickel, iron, copper, cobalt, manganese, zinc, lead and platinum.

6. The method of claim 1, wherein the metal ion is aluminum.

7. The method of claim 1, wherein the monitoring the level of one or more metal ions is carried out using a variable-wavelength UV-Vis detector.

8. The method of claim 1, wherein the solvent is water.

9. The method of claim 1, wherein the ammonium salt comprises at least one compound selected from the group consisting of cetyltrimethyl ammonium bromide, octadecyltrimethyl ammonium bromide, pentadecyltrimethyl ammonium chloride, dodecyltrimethyl ammonium chloride, decyltrimethyl ammonium bromide, octyltrimethyl ammonium bromide, hexyltrimethyl ammonium bromide, butyltrimethyl ammonium chloride, benzyltrimethyl ammonium chloride, diethyldimethyl ammonium chloride, dioctyldimethyl ammonium bromide, tetrabutyl ammonium chloride, tetrapropyl ammonium chloride, tetraethyl ammonium chloride, tetramethyl ammonium chloride, and cetyltrimethyl ammonium phosphate and a mixture thereof.

10. The method of claim 1, wherein the ammonium salt is cetyltrimethyl ammonium bromide.

11. The method of claim 1, wherein the chelating agent is selected from the group consisting of eriochrome cyanine R (ECR), chrome azurol-S, 8-hydroxyquinoline derivatives, 1,2-dihydroxy-3,5-benzene disulfonic acid disodium salt (tiron), hydroxy-2-(2-hydroxyphenylazo)benzene, 5-chloro-2-hydroxy-3-(2,4-dihydroxyphenylazo)benzenesulfonic acid (lumogallion), pyrocatechol violet (PV) and a mixture thereof.

12. The method of claim 1, wherein the chelating agent is eriochrome cyanine R.

13. The method of claim 1, wherein the weight percentage of the chelating agent to the metal detection reagent is in a range of about 0.0001 to about 0.01 percent.

14. The method of claim 1, wherein the weight percentage of the chelating agent to the metal detection reagent is in a range of about 0.001 to about 0.01 percent.

15. The method of claim 1, wherein the weight percentage of the chelating agent to the metal detection agent is in a range of about 0.002 to about 0.01 percent.

16. The method of claim 1, wherein the weight percentage of the ammonium salt to the metal detection agent is in a range of about 0.002 to about 0.01 percent.

17. The method of claim 1, wherein the amount of the ammonium salt is substantially equal to or greater than the chelating agent.

18. The method of claim 1, the metal detection reagent further comprises a pH-controlling agent.

19. The method of claim 18, wherein the pH-controlling agent is selected from the group consisting of acetic acid, citric acid, sulfuric acid, nitric acid, hydrochloric acid, ammonium hydroxide, sodium hydroxide and potassium hydroxide or a mixture thereof.

20. The method of claim 18, wherein the metal detection reagent has a pH in a range of about 1 to about 3, when the pH-controlling agent is acidic.
